(19) United States
(12) Patent Application Publication
(10) Pub. No.: US 2023/0237714 A1
(43) Pub. Date: Jul. 27, 2023

(54) METHOD FOR CONTROLLING APPETITE USING SMART GLASSES AND APPARATUS THEREFOR

(71) Applicant: Minju Kang, Seoul (KR)

(72) Inventor: Minju Kang, Seoul (KR)

(21) Appl. No.: 18/190,034

(22) Filed: Mar. 24, 2023

(51) Int. Cl. G06T 11/00; G06T 7/70; G06T 7/90; G06V 20/68

(52) U.S. Cl. CPC G06T 11/001; G06T 7/70; G06T 7/90; G06V 20/68; G06T 2207/10024

(57) ABSTRACT

Disclosed is a method for controlling appetite using smart glasses having camera and display functions, the method including: by an image processing apparatus, receiving an image captured by the camera from the smart glasses; determining whether a food image exists in the received image and extracting the food image; generating an overlay image in which the color of the extracted food image is converted; and transmitting the generated overlay image to the smart glasses so that the transmitted overlay image is overlaid on the image and displayed. According to the present disclosure, it is possible to control appetite using smart glasses to change the color of food included in the field of view.

### FIELD OF THE INVENTION

The present disclosure relates to a method for controlling appetite and, more particularly, to a method for controlling appetite using smart glasses and an apparatus therefor.

### DESCRIPTION OF THE PRIOR ART

In the field of color psychology, studies are being conducted on the relationship between color and human emotions and behaviors, and one of them is a study that the color of food can affect food preference and even appetite (Jaepil Choi, Goeun Choi, and Hangbong Kang [2010], Color transformation of food images based on user sensibility, Proceedings of the Korea Information Processing Society Conference, Vol. 17, No. 1. 510-513). In addition, in another study, with regard to the appetite response according to the color, the highest appetite response was shown in the colors in a range from red to orange, and the appetite was suppressed in the colors in a range from yellow-green and blue to purple.

Based on the results of these studies, glasses with blue lenses were sold as diet glasses. However, if the color of the lens is always blue, it was difficult to wear these glasses in everyday life, and there was also great discomfort in conversing with people or being aware of the surroundings during meals.

### Patent Literature

Patent Publication No. 10-2020-0122754

### Non-Patent Literature

Jaepil Choi, Goeun Choi, and Hangbong Kang (2010). Color transformation of food images based on user sensibility. Proceedings of the Korea Information Processing Society Conference, Vol. 17, No. 1.

### SUMMARY OF THE INVENTION

The present disclosure has been made in order to solve the above-mentioned problems in the prior art and an aspect of the present disclosure is to provide a method for helping control appetite by converting the color of food included in the field of view using smart glasses, and an apparatus therefor.

Specifically, an aspect of the present disclosure is to provide a method for helping control appetite by suppressing appetite by converting the color of food to a blue-based color, which is a color that suppresses appetite, when viewing food through smart glasses, and an apparatus therefor.

In addition, an aspect of the present disclosure is to provide a method for determining the type of food using a neural network model, etc., to conveniently provide calorie information of food without a separate manipulation by the user and for further helping control appetite and diet by providing body information such as the user's weight or blood sugar, and an apparatus therefor.

The technical problem to be solved by the present disclosure is not limited to the technical problem mentioned above, and another technical problem not mentioned will be clearly understood by those skilled in the art from the contents described in this specification.

In accordance with a first aspect of the present disclosure, there is provided a method for controlling appetite using smart glasses having camera and display functions, the method may include, by an image processing apparatus, receiving an image captured by the camera from the smart glasses; determining whether a food image exists in the received image and extracting the food image; generating an overlay image in which the color of the extracted food image is converted; and transmitting the generated overlay image to the smart glasses so that the transmitted overlay image is overlaid on the image and displayed.

In accordance with a second aspect of the present disclosure, there is provided an apparatus including a processor; and a memory, and the apparatus may perform a specific operation for controlling appetite of a wearer of smart glasses having camera and display functions, wherein the specific operation may include: receiving an image captured by the camera from the smart glasses; determining whether a food image exists in the received image and extracting the food image; generating an overlay image in which the color of the extracted food image is converted; and transmitting the generated overlay image to the smart glasses so that the transmitted overlay image is overlaid on the image and displayed.

In accordance with a third aspect of the present disclosure, there is provided a computer-readable storage medium that stores instructions configured to cause an apparatus including a processor, when executed by the processor, to implement a specific operation for performing appetite control using smart glasses having camera and display functions, wherein the specific operation may include receiving an image captured by the camera from the smart glasses; determining whether a food image exists in the received image and extracting the food image; generating an overlay image in which the color of the extracted food image is converted; and transmitting the generated overlay image to the smart glasses so that the transmitted overlay image is overlaid on the image and displayed.

Preferably, the overlay image may be the extracted food image converted into a blue-based color.

Preferably, the extracting of the food image may include detecting a gaze direction of the wearer of the glasses and extracting the food image existing in an area within a predetermined range in the detected gaze direction.

Preferably, the method or the specific operation may further include: transmitting, to the wearer of the glasses, a message requesting confirmation whether an area where the overlay image is displayed corresponds to food.

Preferably, the extracting of the food image may further include determining a name of food corresponding to the extracted food image.

Preferably, the method or the specific operation may further include: transmitting information on the determined name of the food to the smart glasses; and transmitting, to the wearer of the glasses, a message requesting confirmation whether the information on the name of the food corresponds to the food existing in the image.

Preferably, the method or the specific operation may further include: deriving calorie information of food stored in a database based on the information on the determined name of the food and transmitting the derived calorie information to the smart glasses.

Preferably, the method or the specific operation may include: receiving body information of the wearer of the glasses; storing the body information; and transmitting the body information to the smart glasses.

Preferably, the body information may be received from the smart glasses, a smart watch, a smartphone, a weight scale, a blood pressure monitor, a pulse monitor, or a blood glucose meter.

According to the present disclosure, it is possible to control appetite using smart glasses.

Specifically, according to the present disclosure, it is possible to help suppress appetite by converting the color of food into a color that suppresses appetite through smart glasses, or to also improve anorexia by converting the color of food into an appetizing color.

In addition, according to the present disclosure, it is possible to conveniently provide calorie information of food.

In addition, according to the present disclosure, it is possible to help control appetite and diet by providing calorie information of food and body information of a user through smart glasses.

Effects obtainable in the present disclosure are not limited to the effects mentioned above, and other effects not mentioned can be clearly understood by those skilled in the art from the contents described in this specification.

### BRIEF DESCRIPTION OF THE DRAWINGS

The above and other aspects, features and advantages of the present disclosure will be more apparent from the following detailed description taken in conjunction with the accompanying drawings, in which:

FIG. 1 is a schematic diagram illustrating a smart glasses system according to the proposed method of the present disclosure.

FIG. 2 is a flowchart illustrating a food color conversion process according to the proposed method of the present disclosure.

FIGS. 3A, 3B and 3C are exemplary diagram illustrating food color conversion according to the proposed method of the present disclosure.

FIG. 4 is a flowchart illustrating a food color conversion process according to the proposed method of the present disclosure.

FIG. 5 is a flowchart illustrating a process for providing food information according to the proposed method of the present disclosure.

FIG. 6 is an exemplary diagram illustrating an apparatus to which the proposed method of the present disclosure can be applied.

### DETAILED DESCRIPTION OF THE EXEMPLARY EMBODIMENTS

The present invention can apply various transformations and can have various embodiments. Hereinafter, specific embodiments will be described in detail based on the accompanying drawings.

The following examples are provided to facilitate a comprehensive understanding of the methods, apparatus and/or systems described herein. However, this is only an example and the present invention is not limited thereto.

In describing the embodiments of the present invention, if it is determined that the detailed description of the known technology related to the present invention may unnecessarily obscure the subject matter of the present invention, the detailed description will be omitted. In addition, terms to be described later are terms defined in consideration of functions in the present invention, which may vary according to the intention or custom of a user or operator. Therefore, the definition should be made based on the contents throughout this specification. The terminology used in the detailed description is only for describing the embodiments of the present invention and should in no way be limiting. Unless expressly used otherwise, singular forms of expression include plural forms. In this description, expressions such as “comprising” or “comprised of” are intended to indicate certain characteristics, numbers, steps, operations, elements, some or combinations thereof, and one or more other than those described, and they should not be interpreted to exclude the existence or possibility of one or more other characteristics, numbers, steps, operations, elements, parts or combinations thereof other than those described.

In addition, terms such as first and second may be used to describe various components, but the components are not limited by the terms, and these terms are only used for the purpose of distinguishing one component from another.

Proposed Method of the Present Disclosure

The present disclosure proposes a method of helping control appetite using smart glasses. More specifically, in the present disclosure, in order to help control appetite, there is provided a method for helping users suppress their appetite and control their diet by identifying whether food is included in an image visible to a user wearing smart glasses through the smart glasses to display an overlay image whose color is converted at a location of the food in the image, and by further providing calorie information of the food and body information of the wearer of the glasses together.

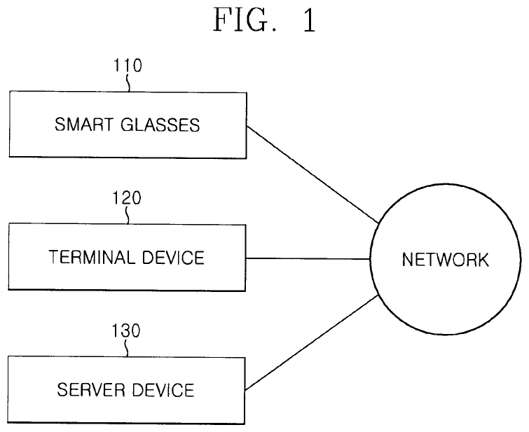

FIG. 1 is a schematic diagram illustrating a smart glasses system according to the proposed method of the present disclosure.

Referring to FIG. 1, a food color conversion process according to the proposed method of the present disclosure may be performed based on smart glasses 110, a terminal device 120, and a server device 130.

At this time, the server device 130 may perform operations by interworking with the terminal device 120 or the smart glasses 110 through a communication network 140. In addition, the terminal device 120 may perform operations by interworking with the smart glasses 110 through the communication network 140.

In the present disclosure, the server device 130 and the terminal device 120 may be simply referred to as a server and a terminal, respectively.

In addition, in the present disclosure, the smart glasses 110 is a device that can be worn near the user's eyes, and may include a transparent display positioned in front of the eyes to provide functions such as augmented reality or virtual reality to the user. The smart glasses 110 may be implemented in various types of devices without being limited to specific types such as glasses and goggles.

In addition, the smart glasses 110 may be equipped with a camera to capture a range similar to a field of view of the wearer at a direction and angle similar to a gaze of the wearer, thereby generating a real-time image that is continuously captured or an image that is discontinuously captured.

More specifically, the smart glasses 110 may exchange data with the terminal device 120 such as a smartphone, a smart pad, or a computer in real-time through the communication network 140 such as Bluetooth, and may be controlled through a software application installed in the terminal device 120 such as a smartphone.

In this specification, the glasses wearer refers to a person wearing the smart glasses 110, and may be referred to as another term such as a user.

In addition, in the present disclosure, as the terminal device 120, various devices capable of receiving and processing data such as images taken from the smart glasses 110 such as a smartphone, a tablet PC, a PDA, a mobile phone, a desktop PC, a notebook PC, a TV, a set-top box, etc., may be used.

In addition, in the present disclosure, the server device 130 may be implemented using one or more physical servers, but the present disclosure is not necessarily limited thereto. In addition, the server device 130 may be configured using a computer processing device such as a desktop computer, a laptop computer, a tablet computer, a smartphone, or the like, or may be implemented in various forms such as a dedicated device.

In addition, as the communication network 140 in FIG. 1, a wired network and a wireless network can be used. Specifically, the communication network 140 may include various communication networks such as a local area network (LAN), a metropolitan area network (MAN), and a wide area network (WAN). In addition, the communication network 140 may include the well-known World Wide Web (WWW). Furthermore, the communication network 140 may be implemented using Bluetooth, infrared (IR) communication, etc., configured to transmit and receive data such as images.

In addition, although FIG. 1 shows that the terminal device 120 is provided in addition to the smart glasses 110 and the server device 130 in the present disclosure, the present disclosure is not necessarily limited thereto. The smart glasses 110 and the server device 130 may be implemented in various forms, such as directly transmitting and receiving data through the communication network 130 without passing through the terminal device 120.

In addition, in the present disclosure, an overlay image refers to an image that can be overlaid on an original image and displayed, and may be overlaid on the original image and displayed while having a predetermined transparency, but the present disclosure is not necessarily limited thereto. The overlay image may be opaquely overlaid on the original image and displayed.

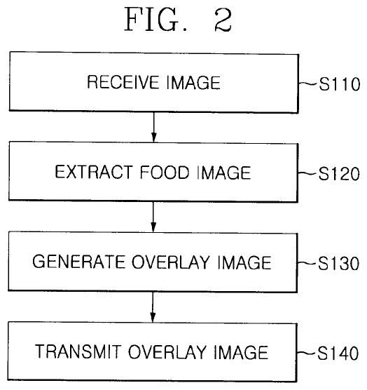

FIG. 2 is a flowchart illustrating a food color conversion process according to the proposed method of the present disclosure. The proposed method of the present disclosure is not limited to the process illustrated in FIG. 2 and may be modified such that some components are excluded or modified or new components are added in the process illustrated in FIG. 2.

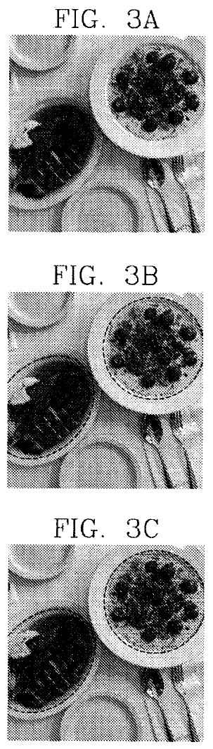

In addition, FIGS. 3A, 3B and 3C are exemplary diagram illustrating food color conversion according to the proposed method of the present disclosure.

First, referring to FIG. 2, the method according to an embodiment of the present disclosure is a method for controlling appetite using smart glasses 110 having camera and display functions, and may include receiving (S110) an image captured by the camera from the smart glasses 110, determining (S120) whether a food image exists in the received image and extracting the food image, generating (S130) an overlay image in which the color of the extracted food image is converted, and transmitting (S140) the generated overlay image to the smart glasses 110 so that the transmitted overlay image is overlaid on the image and displayed.

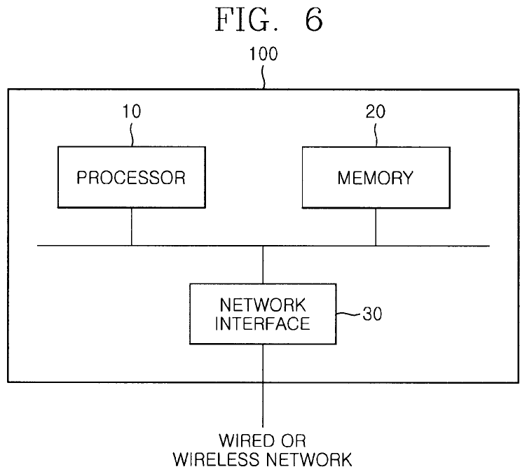

Here, the method shown in FIG. 2 may be performed, for example, by an image processing apparatus, and furthermore, the image processing apparatus may be implemented by including FIG. 6 and a computing device described later in relation to FIG. 6. For example, the image processing apparatus may include a processor 10, and the processor 10 may execute instructions configured to implement an operation for processing the image.

Hereinafter, the present disclosure will be described in more detail in the series of operations.

First, in operation S110, the image processing apparatus receives an image captured by the camera from the smart glasses 110.

At this time, in the present disclosure, the image processing apparatus may be the terminal device 120, but the present disclosure is not necessarily limited thereto. In addition, the image processing apparatus may be the server device 130, and may be configured in various forms such as a module attached to the smart glasses 110 or a module including an arithmetic function built into the smart glasses 110.

Accordingly, the image processing apparatus may receive an image captured by the camera included in the smart glasses 110 through the communication network 140 such as Bluetooth.

Next, in operation S120, the image processing apparatus determines whether a food image exists in the received image, and extracts the food image.

More specifically, as shown in FIGS. 3A and 3B, the image processing apparatus may use a neural network model learned in advance with respect to the image (e.g., FIG. 3A) received from the smart glasses 110 to determine whether the food image is included and extract the food image (e.g., FIG. 3B).

At this time, the neural network model may be implemented and learned using various neural network models such as convolutional neural network (CNN) and recursive neural network (RNN), but the present disclosure is not necessarily limited thereto. In addition, it is also possible to determine and extract the food image in various ways, such as identifying and extracting the food image through similarity analysis in comparison with a database containing one or more food images prepared in advance.

Here, in the extracting of the food image, the image processing apparatus may detect a gaze direction of the glasses wearer, and extract a food image existing in an area within a predetermined range in the detected gaze direction.

For a more specific example, the smart glasses 110 may detect the gaze direction of the glasses wearer through sensor data of the glasses wearer's eyes, and identify and extract a food image by being limited to an area within a predetermined range with respect to the gaze direction of the glasses wearer among the entire area of the image captured by the camera. Through this, it is possible to reduce computing resources required for identifying and processing the food image, and to perform real-time processing even in the image processing apparatus having limited processing capability.

To this end, the smart glasses 110 may separate only an area corresponding to the predetermined range with respect to the gaze direction of the glasses wearer from the image captured by the camera and transmit the separated area to the image processing apparatus. Alternatively, the smart glasses 110 may transmit information about the predetermined range corresponding to the glasses wearer to the image processing apparatus together with the image.

Furthermore, in the present disclosure, it is possible to adaptively adjust the predetermined range according to each user.

More specifically, since the range that can be recognized based on the gaze direction may be different for each user, in the present disclosure, the predetermined range with respect to the gaze direction for each user may be configured in advance in an initial configuration operation. Alternatively, in the process of using the smart glasses 110, the predetermined range may be adaptively adjusted depending on each user while measuring the range recognized by each user with respect to the gaze direction once or multiple times in consideration of a predetermined measurement condition, etc.

In addition, in the extracting of the food image, the image processing apparatus may determine the name of food corresponding to the extracted food image.

In this regard, the image processing apparatus may transmit information on the determined name of the food to the smart glasses, and transmit, to the glasses wearer, a message requesting confirmation whether the information on the name of the food corresponds to the food existing in the image.

Furthermore, the image processing apparatus may derive calorie information of food stored in a database based on the information on the determined name of the food and transmit the derived calorie information to the smart glasses 110.

Next, in operation S130, the image processing apparatus may generate an overlay image in which the color of the extracted food image is converted.

More specifically, the overlay image may be generated by converting the extracted food image into a blue-based color so as to help the glasses wearer control their appetite and diet.

Subsequently, in operation S140, the image processing apparatus may transmit the generated overlay image to the smart glasses 110 so that the transmitted overlay image is overlaid on the image and displayed (e.g., FIG. 3C).

At this time, the image processing apparatus may transmit the information on the determined name of the food together with the overlay image to the smart glasses 110 to be displayed. Furthermore, the image processing apparatus may also derive the calorie information of the food stored in the database based on the information on the determined name of the food, and transmit the derived calorie information to the smart glasses 110 to be displayed together.

In addition, the image processing apparatus may receive and store body information of the glasses wearer, which is input through an app or the like executed on a smartphone or the like, and transmit and provide the body information to the smart glasses.

More specifically, the body information may be received from the smart glasses 110, smart watch, smartphone, weight scale, blood pressure monitor, pulse monitor, or blood glucose meter, but the present disclosure is not necessarily limited thereto.

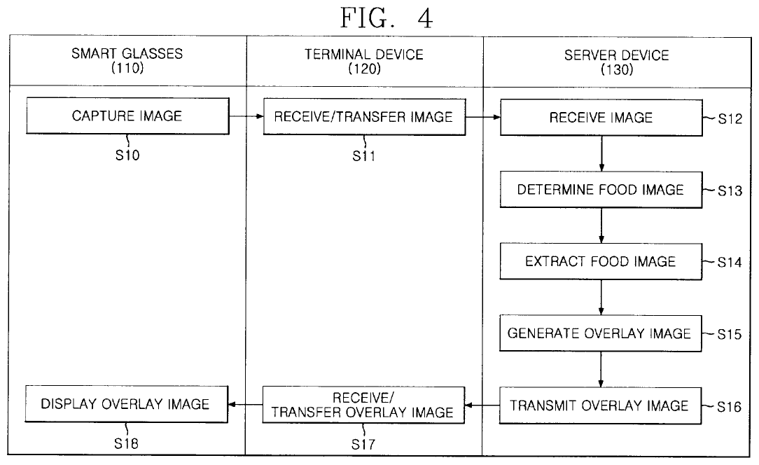

In addition, FIG. 4 is a flowchart illustrating a food color conversion process according to the proposed method of the present disclosure. The proposed method of the present disclosure is not limited to the process illustrated in FIG. 4 and may be modified such that some components are excluded or modified or new components are added in the process illustrated in FIG. 4.

In addition, in FIG. 4, a method of processing the image in the server 130 based on the image captured by the smart glasses 110 is described as an example, but the present disclosure is not necessarily limited thereto, and the method of processing the image may be implemented in various ways, such as processing the image by the terminal 120.

At this time, the smart glasses 110 may include a camera, and the camera may capture a range similar to the wearer's field of view at a direction and angle similar to the gaze in operation S10.

The smart glasses 110 may transmit the image captured in real-time by the camera to the terminal 120, and the terminal 120 may transmit information on the captured image in real-time to the server 130 in operation S11.

Subsequent, the server 130 may receive real-time image information from the terminal 120 in operation S12, determine whether a food image exists in a part of the received image using a neural network model in operation S13, and extract the food image when the food image exist in operation S14.

At this time, the server 130 may have a database, and the database may store/manage images of food, and additionally store/manage the name, type, calories, and other nutritional information of food corresponding to the image of food.

In addition, the server 130 may learn the food image using the neural network model, and discriminate and extract the food image using the learned neural network model.

Accordingly, the server 130 may generate an overlay image in which the color of an area corresponding to the extracted food image is converted in operation S15.

For example, an overlay image whose color is converted into a blue-based color may be generated. The overlay image of the blue-based color may suppress appetite for food. As another example, an overlay image whose color is converted into a red-based color may be generated. The overlay image of the red-based color may stimulate appetite. The glasses wearer may configure an appetite control mode by stimulating or suppressing appetite through the terminal 120.

Subsequently, the server 130 may transmit the color-converted overlay image to the terminal 120 in operation S16, and the terminal 120 may transmit the overlay image to the smart glasses 110 in operation S17.

At this time, the smart glasses 110 may include a display, and the display may display the overlay image transmitted from the terminal 120 in operation S18.

Here, the server 13 may calculate an area where the overlay image is to be displayed based on data transmitted from the smart glasses 110 and transmit the coordinates of the area to be displayed to the smart glasses 110.

Furthermore, the smart glasses 110 may additionally include an eye sensor. The server 130 may receive and analyze information measured by the eye sensor to detect the gaze direction of the glasses wearer.

The server 130 may determine whether the food image exists in a part of the image corresponding to the area within the predetermined range in the gaze direction of the glasses wearer based on the detected gaze direction, and extract the food image present in the area.

For example, the glasses wearer may configure a range of an area to determine whether the food image exists through the terminal 120. As another example, the server 130 may automatically configure an optimal range by calculating the range of the area to determine whether the food image exists based on a gaze range and gaze movement of the glasses wearer.

In addition, the smart glasses 110 may additionally include an input unit. For example, the input unit may be a mechanical button provided on the body of the smart glasses 110, a sensor that recognizes a touch, or a device that recognizes a voice.

The server 130 may transmit, to the glasses wearer, a confirmation requesting whether the area where the overlay image is displayed corresponds to food. The glasses wearer may input a response to the confirmation request of the server 130 through the input unit.

Alternatively, the glasses wearer may input a response to the confirmation request of the server 130 using the input unit such as a touch screen provided in the terminal 120 such as a smartphone.

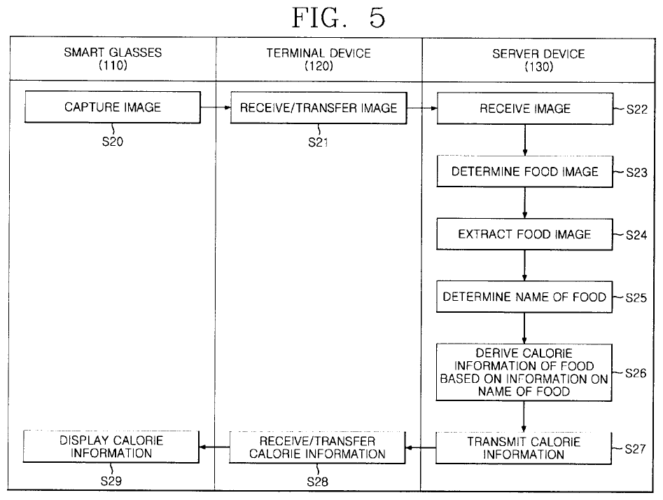

FIG. 5 is a flowchart illustrating a process for providing food information according to the proposed method of the present disclosure. The proposed method of the present disclosure is not limited to the process illustrated in FIG. 5 and may be modified such that some components are excluded or modified or new components are added in the process illustrated in FIG. 5.

In addition, in FIG. 5, a method of processing the image by the server 130 based on the image captured by the smart glasses 110 is described as an example, but the present disclosure is not necessarily limited thereto. The method of processing the image may be implemented in various ways, such as processing the image by the terminal 120.

At this time, the smart glasses 110 may include a camera, and the camera may capture a range similar to the wearer's field of view at a direction and angle similar to the gaze of the wearer in operation S20.

The smart glasses 110 may transmit an image captured in real-time by the camera to the terminal 120, and the terminal 120 may transmit information on the captured image in real-time to the server 130 in operation S21.

Subsequently, the server 130 may receiver real-time image information from the terminal 120 in operation S22, determine whether a food image exists in a part of the received image using a neural network model in operation S23, and extract the food image when the food image exists in operation S24.

Furthermore, when determining that the food image exists in the part of the received image and extracting the food image, the server 130 may additionally determine the name of food corresponding to the food image using the neural network model or the like in operation S25.

Accordingly, the server 130 may transmit information on the determined name of the food, and transmit, to the glasses wearer, a request to confirm whether the determined name of the food corresponds to the name of the food present in the received image. The glasses wearer may input a response to the confirmation request of the server 130 through the input unit.

In addition, the server 130 may derive calorie information and/or other nutritional information of food stored in the database based on the information on the determined name of the food in operation S26, and transmit the derived information in operation S27.

In addition, the database of the server 130 may additionally store/manage color information that stimulates or suppresses appetite according to food.

Accordingly, the server 130 may generate an overlay image whose color is converted according to an appetite control mode configured by the glasses wearer, based on appetite control color information according to food in the database.

Furthermore, the server 130 may additionally receive body information of the glasses wearer, store the received body information, and transmit the stored body information.

At this time, the server 130 may receive weight, body fat mass, blood pressure, pulse, blood sugar, number of steps, amount of exercise, or other body information from the smart glasses, smart watch, smartphone, weight scale, blood pressure monitor, pulse monitor, blood glucose meter, or other measurement devices.

Apparatus to Which the Proposed Method of the Present Disclosure Can Be Applied

FIG. 6 is an exemplary diagram illustrating an apparatus 100 to which the proposed method of the present disclosure can be applied.

Referring to FIG. 6, an apparatus 100 may be configured to implement the proposed method of the present disclosure and a food color conversion process according to the method. For example, the apparatus 100 may function as an image processing apparatus according to the present disclosure, may be configured as a separate device, or may be configured using one or more of the smart glasses 110, the terminal device 120, and the server device 130.

For example, the apparatus 100 to which the proposed method of the present disclosure can be applied may include a network device such as a repeater, a hub, a bridge, a switch, a router, and a gateway, and the terminal device 120 may include a computer device such as a desktop computer, a mobile terminal such as a smart phone, a portable device such as a laptop computer, and home appliances such as a digital TV. The smart glasses 110 may include a camera, a display, and an eye sensor, and may further include an input unit such as a button, a touch sensor, and a microphone.

The memory 20 may be connected to the processor 10 during operation, and may store programs and/or instructions for processing and controlling the processor 10, and may store data and information used in the present disclosure, control information necessary for data and information processing according to the present disclosure, and temporary data generated during data and information processing.

The memory 20 may be implemented as a storage device such as read only memory (ROM), random access memory (RAM), erasable programmable read only memory (EPROM), electrically erasable programmable read-only memory (EEPROM), flash memory, static RAM (SRAM), hard disk drive (HDD), solid state drive (SSD), etc.

The processor 10 may be operatively connected to the memory 20 and the network interface 30, and control the operation of each module in the apparatus 100. In particular, the processor 10 may perform various control functions for performing the proposed method of the present disclosure. The processor 10 may also be called a controller, a microcontroller, a microprocessor, a microcomputer, or the like. The proposed method of the present disclosure may be implemented by hardware, firmware, software, or a combination thereof In the case of implementing the present disclosure using hardware, an application specific integrated circuit (ASIC) or a digital signal processor (DSP), a digital signal processing device (DSPD), a programmable logic device (PLD), a field programmable gate array (FPGA), and the like, which are configured to perform the present disclosure, may be provided in the processor 10. On the other hand, when implementing the proposed method of the present disclosure using firmware or software, the firmware or software may include instructions related to modules, procedures, or functions that perform functions or operations necessary to implement the proposed method of the present disclosure. When the instructions are stored in the memory 20 or stored in a computer-readable recording medium (not shown) separate from the memory 20 and executed by the processor 10, the instructions may be configured so that the apparatus 100 implements the proposed method of the present disclosure.

In addition, the apparatus 100 may also include a network interface device 30. The network interface device 30 may be connected to the processor 10 during operation, and the processor 10 may control the network interface device 30 to transmit or receive wireless/wired signals carrying information and/or data, signals, messages, etc., through a wireless/wired network. The network interface device 30 may support various communication standards such as IEEE 802 series, 3GPP LTE(-A), 3GPP 5G, etc., and transmit and receive control information and/or data signals according to the communication standards. The network interface device 30 may be implemented outside the apparatus 100 as needed.

The embodiments described above are those in which the components and features of the present disclosure are combined in a predetermined form. Each component or feature should be considered optional unless explicitly stated otherwise. Each component or feature may be implemented in a form not combined with other components or features. It is also possible to configure an embodiment of the present disclosure by combining some components and/or features. The order of operations described in the embodiments of the present disclosure may be changed. Some components or features of one embodiment may be included in another embodiment, or may be replaced with corresponding components or features of another embodiment. It is obvious that claims that do not have an explicit citation relationship in the claims can be combined to form an embodiment or can be included as new claims by amendment after filing.

### INDUSTRIAL APPLICABILITY

The present disclosure can be applied to smart glasses.

### Description of Reference Numerals

110: smart glasses

120: terminal device

130: server device

What is claimed is:

1. A method for controlling appetite using smart glasses having camera and display functions, the method comprising:
by an image processing apparatus, receiving an image captured by the camera from the smart glasses;
determining whether a food image exists in the received image and extracting the food image;
generating an overlay image in which the color of the extracted food image is converted; and
transmitting the generated overlay image to the smart glasses so that the transmitted overlay image is overlaid on the image and displayed.

2. The method of claim 1, wherein the overlay image is the extracted food image converted into a blue-based color.

3. The method of claim 1, wherein the extracting of the food image comprises detecting a gaze direction of the wearer of the glasses, and extracting the food image existing in an area within a predetermined range in the detected gaze direction.

4. The method of claim 1, further comprising transmitting, to the wearer of the glasses, a message requesting confirmation whether an area where the overlay image is displayed corresponds to food.

5. The method of claim 1, wherein the extracting of the food image further comprises determining a name of food corresponding to the extracted food image.

6. The method of claim 5, further comprising:
transmitting information on the determined name of the food to the smart glasses; and
transmitting, to the wearer of the glasses, a message requesting confirmation whether the information on the name of the food corresponds to the food existing in the image.

7. The method of claim 5, further comprising:
deriving calorie information of food stored in a database based on the information on the determined name of the food; and
transmitting the derived calorie information to the smart glasses.

8. The method of claim 1, further comprising:
receiving body information of the wearer of the glasses;
storing the body information; and
transmitting the body information to the smart glasses.

9. The method of claim 8, wherein the body information is received from the smart glasses, a smart watch, a smartphone, a weight scale, a blood pressure monitor, a pulse monitor, or a blood glucose meter.

10. An apparatus that comprises a processor and a memory and performs specific operations for controlling appetite of a wearer of smart glasses having camera and display functions, wherein the specific operations comprise:
receiving an image captured by the camera from the smart glasses;
determining whether a food image exists in the received image and extracting the food image;
generating an overlay image in which the color of the extracted food image is converted; and
transmitting the generated overlay image to the smart glasses so that the transmitted overlay image is overlaid on the image and displayed.

11. A computer-readable storage medium that stores instructions configured to, when executed by a processor, cause an apparatus comprising the processor to implement specific operations for performing appetite control using smart glasses having a camera and display functions, wherein the specific operations comprise:
receiving an image captured by the camera from the smart glasses;
determining whether a food image exists in the received image and extracting the food image;
generating an overlay image in which the color of the extracted food image is converted; and
transmitting the generated overlay image to the smart glasses so that the transmitted overlay image is overlaid on the image and displayed.
